(19) United States
(12) Patent Application Publication
(10) Pub. No.: US 2012/0293189 A1
(43) Pub. Date: Nov. 22, 2012

(54) NOVEL METHOD AND DEVICE FOR WHOLE-CELL BACTERIAL BIO-CAPACITOR CHIP FOR DETECTING CELLULAR STRESS INDUCED BY TOXIC CHEMICALS

(75) Inventors: Anjum Qureshi, Istanbul (TR); Yasar Gurbuz, Istanbul (TR); Javed Hussain Niazi Kolkar Mohammed, Istanbul (TR); Saravan Kallempudi, Istanbul (TR)

(73) Assignee: Sabanci Universitesi, Istanbul (TR)

(21) Appl. No.: 13/473,557

(22) Filed: May 16, 2012

Related U.S. Application Data
(60) Provisional application No. 61/487,225, filed on May 17, 2011.
(60) Provisional application No. 61/488,693, filed on May 20, 2011.

(51) Int. Cl. G01R 27/26; H01L 21/02; H01L 29/66; C12N 11/14; B82Y 15/00

(52) U.S. Cl. 324/658; 257/414; 435/176; 438/49; 257/E29.166; 977/742; 977/902; 257/E21.011

(57) ABSTRACT

The present invention is directed to methods and a bio-capacitor sensing device for the detection of toxic chemicals using bacteria. The sensing platform comprises gold interdigitated capacitor with a defined geometry, a layer of carboxy-CNTs immobilized with viable E. coli cells as sensing elements. Also included are methods of making the bio-capacitor device and methods for detecting toxic chemicals that induce cellular stress response. The present innovation discloses the development of a bio capacitor chips immobilized with carboxy-CNTs tethered E. coli bacteria. In addition, the present invention also includes determination of behavior and characteristics of chemically stimulated bacteria on biochip using electric field including frequency and/or amplitude as controlling parameters.

### CROSS-REFERENCE TO RELATED APPLICATIONS

This application claims priority to U.S. Provisional Application No. 61/487,225, filed on May 17, 2011; and U.S. Provisional Application No. 61/488,693, filed on May 20, 2011; each of which is hereby incorporated by reference for all purposes.

### FIELD OF INVENTION

The present invention generally relates to the development of whole-cell bacterial bio-capacitor chip technology. More particularly to methods and a whole-cell E. coli bio-capacitor chip device for determining cellular stress induced by toxic chemicals at bacteria-capacitor interface.

### BACKGROUND

Microorganisms, such as bacteria can be used as biological sensing elements to determine the toxicity nature of a variety of chemicals. Sensing the toxic nature of chemicals on bacterial cells enables predicting chemicals' potential to induce toxicity in other living species including humans. A majority of chemicals are toxic in nature to living cells. These can be screened and predicted in mixtures. Chemicals derived from pharmaceutical preparations, drugs, defense agents, contaminated environmental and food samples typically exhibit detrimental effects by inducing cellular damages, such as oxidative, genotoxic, and metabolic stresses and thus are harmful to living organisms.

Living cells typically are known to be utilized that potentially allow assessing toxicological risk and to determine the toxic nature of chemicals when they are exposed. Bacterial cells can be an ideal choice as biological recognition elements because they are known to respond to the external stress (stimuli), such as by toxic chemicals that lead to altered cellular dynamics, including metabolism, growth and cell surface charge distribution. Such responses can be utilized to predict the toxicity of chemicals. The toxicity response of bacterial cells is often determined in terms of various stress responses. Typically, the stress responses in bacteria are classified into different types based on the nature of the chemical compound used to induce toxicity. For example, chemicals that induce various cellular toxicity responses through different modes such as by (i) metabolic/acid toxicity induced by chemicals such as, acetic acid, lactic acid organic calcium salts, propionate, formate and drugs that influence intracellular accumulation of anions; (ii) oxidative toxicity induced by chemicals that produce reactive oxygen species (ROS) such as H₂O₂, hydroxyl radical (.OH), superoxide anion (O₂⁻), organic hydrogen peroxide (ROOH), peroxynitrite (OONO) and nitric oxide (NO); and (iii) Osmotic stress induced by high concentrations of solutes include high levels of NaCl, osmolytes in the cytosol of cells subjected to osmotic stress, such as by carnitine, trihalose, glycerol, sucrose, proline, mannitol, and glycine-betain and others induce genotoxic stress, and various cellular stress responses.

There are a variety of known methods to detect and measure the toxicity or the cell-killing property of a toxicant. Conventional methods follow the cellular metabolic rate (e.g., tetrazolium salt cleavage), and the activity of a cytoplasmic enzyme (e.g., lactate dehydrogenase). The neutral red uptake assay (NR) and the total cellular protein assay are also the two principal methodologies for testing toxicity. Other cytotoxicity methods involve the detection of pH changes in the neighborhood of cultured cells by a silicon microphysiometer and the measurement of the barrier function of a cell layer (transcellular resistance) upon exposure to test compounds.

Although the above methods are noninvasive methods that present quantitative measurements, a common limitation is that they require cell layers grown on membrane inserts or suspension in culture medium. These techniques are typically not suitable for testing toxic gases (defense agents) and the living cells on to which the toxic chemicals are to be tested are sacrificed or require nutrient medium to be present all the time during the tests.

Other methods include studying cytotoxicity by cellular and video imaging analysis. However, these disadvantageously require extensive data processing and only provide semi-quantitative results. One example of a commercially available microbial based toxicity screening method is available under the name Microtox®, which utilizes luminescent bacteria for measuring the effect of toxicants. Such techniques can be more susceptible to physical factors such as thermal, partial pressure and pH to which luminescence will develop and typically is not suitable for testing toxic gases (defense agents) and these are also required to depend on bacterial cells to express luminescent gene product and a luminometer.

Another example includes a method utilizing commercially available laboratory equipment manufactured by Applied BioPhysics Inc. (ABP), which produces Electric Cell-substrate Impedance Sensing (ECIS) equipment. This method utilizes electrodes and counter electrodes that are “joined” by a culture medium to measure impedance response. There are major disadvantages with this type of system, since the culture medium or any other liquid medium, generally is known to alter the behavioral response of cells. In such cases, it can be difficult to distinguish the responses induced by the chemical agent in the context, from that of a complex mixture of other chemicals present in the nutrient medium. Typically, ECIS of ABP requires the culture/liquid medium should be present in order to obtain cellular response, which can interfere with actual response of a target chemical in the nutrient mixture.

Therefore, while these aforementioned methods can be useful, they are often disadvantageously unable to detect the toxicity on living cells by monitoring the damages on cell surface caused that is specifically induced by toxic chemicals, including gases in absence of any interfering media, such as culture/liquid medium.

At the present time, there exists a need for a method and device that can measure and detect the toxicity of chemicals and impact of such chemicals on humans. Further, it would be advantageous to have such methods and device to be used to screen various chemicals, toxic gases, pharmaceuticals, drugs, defense agents, environmental and food samples for the determination of chemicals' potential to cause cytotoxicity. Moreover, these methods and apparatus will be cost effective, have high sensitivity and selectivity, and have fast response. Such methods and devices will have numerous applications in the medical and clinical diagnosis, environmental monitoring, food industry, defense and protection and are applicable for many other diagnostic, biotechnical and scientific purposes.

### SUMMARY

The present invention is directed to methods and a device for high accuracy determining cellular stress induced by toxic chemicals at bacteria-capacitor interface that meets these needs. The methods and device according to the present invention, can be used in determining cellular stress induced by toxic chemicals at bacteria-capacitor interface.

The present invention is directed to a bio-capacitor sensing device for the detection of a target chemical, the sensing device comprising: a capacitor comprising a substrate and a metal deposit layer on the substrate; a layer of carboxylated carbon nanotubes (carboxy-CNTs); and viable cells, wherein the viable cells are immobilized to the layer of carbon nanotube (CNT). The viable cells are sensing elements that are capable of adapting to respond with the target chemical and the viable cells can be monitored for stress imposed by the target chemical on the viable cells with no interfering nutrient/culture medium.

The substrate is selected from the group consisting of silicon, glass, melted silica, and plastics. Preferably, the substrate is silicon.

The metal deposit layer on the substrate comprises at least one electrode. The electrode is a material selected from the group consisting of gold, silver, platinum, palladium, copper and indium tin oxide (ITO). More preferably, the electrode is gold.

Preferably, the capacitor is a gold interdigitated capacitor.

The layer of carbon nanotubes can be carboxylated multiwalled carbon nanotubes (carboxy-CNTs).

The viable cells can be selected from the group consisting of mammalian cells, bacterial cells and tissue cells of specific function. Preferably, the viable cells are bacterial cells. The bacterial cells may be any strain of bacterial cells comprising Escherichia coli DH5α, K-12, Salmonella, Pseudomonas, and Bacillus species. Preferably, the bacterial cells are Escherichia coli.

The target chemical can be selected from the group consisting of, acetic acid, lactic acid organic calcium salts, propionate, formate, drugs that influence intracellular accumulation of anions; oxidative toxicity induced by chemicals that produce reactive oxygen species (ROS), H₂O₂, hydroxyl radical (.OH), superoxide anion (O₂⁻), organic hydrogen peroxide (ROOH), peroxynitrite (OONO), nitric oxide (NO); osmotic stress induced by high concentrations of solutes, NaCl, osmolytes in the cytosol of cells; carnitine, trihalose, glycerol, sucrose, proline, mannitol, glycine-betain and others that induce genotoxic stress.

The present invention is directed to a bio-capacitor sensing device for the detection of a target chemical, the sensing device comprising: a gold interdigitated capacitor comprising a substrate and a gold interdigitated layer on the substrate; a layer of carboxylated multiwalled carbon nanotubes (carboxy-CNTs); and viable bacterial cells, wherein the viable bacterial cells are immobilized to the layer of carbon nanotube (CNT), whereby the viable bacterial cells are sensing elements that are capable of adapting to respond with the target chemical, wherein the viable bacterial cells can be monitored for stress imposed by the target chemical on the viable bacterial cells under dry conditions with no other interfering liquid nutrient/culture medium.

The present invention is directed to a method of detecting the presence, measuring the amount or verifying a target chemical of interest in a test sample, wherein the method is characterized using the bio-capacitor sensing device.

The present invention is directed to method of quantitatively detecting a target chemical of interest, the method comprising the steps of: exposing a test sample to the bio-capacitor device, wherein the test sample contains the target chemical of interest, whereby the test sample is capable of inducing a cellular stress response to the bio-capacitor device; applying a potential profile with an alternative current (AC) frequency to the bio-capacitor device; monitoring the cellular stress response from the bio-capacitor device by measuring the change in surface impedance/capacitance of the bio-capacitor device by non-Faradaic electrochemical impedance spectroscopy (nFEIS), wherein the cellular response correlates with the presence of the target chemical of interest without the interference of nutrient/culture medium. The bio-capacitor device can have cells present on the device, and the test sample is capable of inducing a cellular stress response to the cells present on the bio-capacitor device.

The target chemical can be a stress agent selected from the group consisting of acetic acid, lactic acid organic calcium salts, propionate, formate, drugs that influence intracellular accumulation of anions; oxidative toxicity induced by chemicals that produce reactive oxygen species (ROS), H₂O₂, hydroxyl radical (.OH), superoxide anion (O₂⁻), organic hydrogen peroxide (ROOH), peroxynitrite (OONO), nitric oxide (NO); osmotic stress induced by high concentrations of solutes, NaCl, osmolytes in the cytosol of cells; carnitine, trihalose, glycerol, sucrose, proline, mannitol, glycine-betain and others that genotoxic stress.

The present invention is directed to a method of quantitatively detecting a target chemical of interest, the method comprising the steps of: A method of exposing a test sample to the bio-capacitor device, wherein the test sample contains the target chemical of interest, whereby the test sample is capable of inducing a cellular stress response to the bio-capacitor device; applying a potential profile with an alternative current (AC) frequency to the bio-capacitor device; monitoring the cellular stress response of the bio-capacitor device by measuring the change in surface impedance/capacitance of the bio-capacitor device by nFEIS under no interfering culture medium, wherein the cellular response correlates with the presence of the target chemical of interest.

A method of producing a bio-capacitor sensing device, the method comprising the steps of: providing a substrate; depositing a metal layer on the substrate to form a capacitor, wherein the metal layer comprises at least one electrode; patterning the metal layer in interdigitated fingers on silicon dioxide substrate, making a capacitor; attaching a layer of carboxylated carbon nanotubes (carboxy-CNTs) to the capacitor to form a carboxy-CNT activated capacitor; immobilizing viable cells to the carboxy-CNT activated capacitor, whereby the viable cells are sensing elements that are capable of adapting to respond with a target chemical, wherein the viable cells can be monitored for stress imposed by the target chemical on the viable cells in absence of interfering culture/nutrient medium.

The substrate is selected from the group consisting of silicon, glass, melted silica, and plastics. Preferably, the substrate is silicon.

The electrode is a material selected from the group consisting of gold, silver, platinum, palladium, copper and indium tin oxide (ITO). Preferably, the electrode is gold. The capacitor is a gold interdigitated capacitor.

The layer of carbon nanotubes are carboxylated multiwalled carbon nanotubes (carboxy-CNTs).

The viable cells can be selected from the group consisting of mammalian cells, bacterial cells and tissue cells of specific function. Preferably, the viable cells are bacterial cells comprising Escherichia coli, K-12, Salmonella, Pseduomonas, and Bacillus species. More preferably, the bacterial cells are Escherichia coli.

These features, advantages and other embodiments of the present invention are further made apparent, in the remainder of the present document, to those of ordinary skill in the art.

### BRIEF DESCRIPTION OF DRAWINGS

These and other features, aspects, and advantages of the present invention will become better understood with reference to the following description, appended claims, and accompanying drawings where:

FIG. 1 illustrates an exemplary schematic diagram of activation of gold interdigitated electrode capacitor chip with carboxy-CNT fictionalization according to the embodiments of the present invention.

FIG. 2 illustrates an exemplary schematic diagram of biofunctionalization of carboxy-CNT activated gold interdigitated (GID) capacitor chip and immobilization of E. coli cells to develop biochips according to the embodiments of the present invention.

FIG. 3 illustrates tapping-mode AFM images (within 4.2×4.2 μm² scan area) of (a) bare GID surface, (b) Line plot surface profile of the selected green line region (1 μm length) in the tapping-mode AFM height image of bare GID surface, (c) 3D AFM topographical map of bare GID surface, (d) Tapping-mode AFM height image of GID surface activated with carboxy-CNTs, (e) Line plot surface profile of the selected green line region (1 μm length) in the tapping-mode AFM height image of GID electrode on capacitor surface activated with carboxy-CNTs, (f) 3D AFM topographical map of carboxy-CNT activated GID surface, and (g) A 2D tapping mode AFM image of a section (scan area 4.2 μm²) of biochip showing immobilized E. coli cells according to an embodiment of the present invention.

FIG. 4 illustrates an exemplary capacitive response of gold interdigitated capacitor chip before and after carboxy-CNT immobilization according to the embodiments of the present invention.

FIG. 5 illustrates an exemplary optical micrographs of gold interdigitated capacitor surface: (I) activated with carboxy-CNTs (control), and carboxy-CNT activated chips immobilized with E. coli with concentrations of (II) 8.7×10⁶ cells and (III) 1.7×10⁷ cells. The rows (a-c, d-f and g-i) indicate optical resolutions of 5×, 10× and 100×, respectively according to the embodiments of the present invention.

FIG. 6 illustrates exemplary capacitive response of biochips immobilized with two different concentrations of E. coli cells on GID surface that was previously activated with carboxy-CNTs. The capacitive responses were observed at a frequency sweep of 50-600 MHz in absence of nutrient/culture medium according to the embodiments of the present invention.

FIG. 7 illustrates a schematic representation of (a) Capacitor array biochip immobilized with viable E. coli cells by tethering with carboxy-CNTs on gold interdigitated electrodes of each capacitor with a defined geometry and dimension; and (b) diagram showing the response of E. coli and surface charge distribution under the applied AC frequency in normal and chemical stress conditions in absence of nutrient/culture medium, according to the embodiments of the present invention.

FIG. 8 illustrates a change in capacitance from E. coli capacitor biochip as a function of applied frequency (300-600 MHz) when exposed to different concentration of acetic acid for (a) 1 h and (b) 3 h in absence of nutrient/culture medium according to the present invention.

FIG. 9 illustrates a change in capacitance with E. coli capacitor biochip as a function of applied frequency (300-600 MHz) when exposed to different concentration of H₂O₂ for (a) 1 h and (b) 3 h in absence of nutrient/culture medium according to the present invention.

FIG. 10 illustrates change in capacitance from E. coli capacitor biochip as a function of applied frequency (300-600 MHz) when exposed to different concentration of NaCl for (a) 1 h and (b) 3 h in absence of nutrient/culture medium according to the present invention.

FIG. 11 illustrates response of E. coli cells (immobilized on CNT activated sensor chip) as a function of different concentrations of (a) acetic acid (acid stress), (b) H₂O₂ (oxidative stress) and (c) NaCl (salt stress) at a constant AC electrical frequency (350 MHz) for 1 and 3 h treatment times in absence of nutrient/culture medium. The inset tables show colour coded values determining the percent relative change in stress levels experienced by E. coli cells. The stress colour code scale indicates the severity of the stress levels in which green represent adaptation/resistance and red represent stress/toxicity according to an embodiment of the present invention.

FIG. 12 illustrates exemplary results of capacitive response of bare GID surface covalently linked with only carboxy-CNTs (shown in black); biochip immobilized with viable 8.7×10⁶ cells (red) and 1.74×10⁷ cells (blue); and heat-killed 1.74×10⁷ cells (green) on GID surface that was previously activated with carboxy-CNTs. The capacitive responses were observed at a frequency range of 300-600 MHz in absence of nutrient/culture medium according to an embodiment of the present invention.

FIG. 13 illustrates an exemplary schematic diagram of a typical cell surrounded by a cloud of charges that constitutes a molecular dipole ‘m’ by two equal and opposite unit charges, separated by a distance ‘r’ on an outer cell surface according to an embodiment of the present invention.

### DETAILED DESCRIPTION OF SPECIFIC EMBODIMENTS

According to the present invention, a new capacitive biochip was developed using carboxy-CNT activated gold interdigitated (GID) capacitors immobilized with E. coli cells for the detection of cellular stress caused by chemicals.

The present invention, according to an embodiment, describes the development of a whole-cell E. coli bio-capacitor chip device for determining cellular stress induced by toxic chemicals at bacteria-capacitor interface. The developed technology also describes fabrication of electronic gold interdigitated electrode (GID) capacitor in conjunction with carboxylated carbon nanotubes (carboxy-CNTs) immobilized with viable bacterial cells as sensing elements (bio-capacitor). The proposed innovation also discloses the surface characteristics of bio-capacitor chip for sensing potential toxic chemicals using model chemicals such as, acetic acid (CH₃COOH) for acid toxicity, hydrogen peroxide (H₂O₂) for oxidative toxicity and sodium chloride (NaCl) for salt stress. The bio-capacitor device and detection methodology is based on non-Faradaic electrochemical impedance spectroscopy (nFEIS). The proposed invention/technology and detection methodology thereof can be used to screen various chemicals, toxic gases, pharmaceuticals, drugs, defense agents, environmental and food samples for the determination of chemicals' potential to cause cytotoxicity.

According to one embodiment, the present invention is directed to a bio-capacitor sensing device for the detection of a target chemical, the sensing device comprising: a capacitor comprising a substrate and a metal deposit layer on the substrate; a layer of carboxylated carbon nanotubes (carboxy-CNTs); and viable cells, wherein the viable cells are immobilized to the layer of carbon nanotube (CNT). The viable cells are sensing elements that are capable of adapting to respond with the target chemical and the viable cells can be monitored for stress imposed by the target chemical on the viable cells in absence of nutrient/culture medium.

Typically, the substrate is selected from the group consisting of silicon, glass, melted silica, and plastics. Preferably, the substrate is silicon.

The metal deposit layer on the substrate comprises at least one electrode in the form of interdigitated fingers. Typically, the electrode is a material selected from the group consisting of gold, silver, platinum, palladium, copper and indium tin oxide (ITO). More preferably, the electrode is gold.

According to example embodiments of the present invention, biosensors fabricated in the form of a chip may also be referred to as a biochip. Bio-capacitor sensing device and biochip can be used interchangeably throughout.

Preferably, the capacitor is a gold interdigitated capacitor. Preferably, the bio-capacitor sensing device provides a sensing platform comprising a gold interdigitated capacitor with a defined geometry.

According to an embodiment of the present invention, the layer of carbon nanotubes preferably are carboxylated multiwalled carbon nanotubes (carboxy-CNTs).

According to yet another embodiment of the present invention, the viable cells can be selected from the group consisting of mammalian cells, bacterial cells and tissue cells of specific function. Preferably, the viable cells are bacterial cells. The bacterial cells may be any strain of bacterial cells comprising Escherichia coli DH5α, K-12, Salmonella, Pseudomonas and Bacillus species. Preferably, the bacterial cells are Escherichia coli.

In yet a preferred embodiment of the present invention, bacterial cells can be an ideal choice as biological recognition elements because they are known to respond to the external stress (stimuli), such as by toxic chemicals that lead to altered cellular dynamics, including metabolism, growth and cell surface charge distribution. Such responses can be utilized to predict the toxicity of chemicals. The toxicity response of bacterial cells is often determined in terms of various stress responses. Typically, the stress responses in bacteria are classified into different types based on the nature of the chemical compound used to induce toxicity.

According to the present invention, the target chemicals typically are chemicals that are stress agents, that can induce various cellular toxicity responses through different modes such as by (i) metabolic/acid toxicity induced by chemicals such as, acetic acid, lactic acid organic calcium salts, propionate, formate and drugs that influence intracellular accumulation of anions; (ii) oxidative toxicity induced by chemicals that produce reactive oxygen species (ROS) such as H₂O₂, hydroxyl radical (.OH), superoxide anion (O₂⁻), organic hydrogen peroxide (ROOH), peroxynitrite (OONO) and nitric oxide (NO); and (iii) Osmotic stress induced by high concentrations of solutes include high levels of NaCl, osmolytes in the cytosol of cells subjected to osmotic stress, such as by carnitine, trihalose, glycerol, sucrose, proline, mannitol, and glycine-betain and others that induce genotoxic stress, and various cellular stress responses.

In yet another preferred embodiment, the present invention is directed to a bio-capacitor sensing device for the detection of a target chemical, the sensing device comprising: a gold interdigitated capacitor comprising a substrate and a gold interdigitated layer on the substrate; a layer of carboxylated multiwalled carbon nanotubes (carboxy-CNTs); and viable bacterial cells, wherein the viable bacterial cells are immobilized to the layer of carbon nanotube (CNT), whereby the viable bacterial cells are sensing elements that are capable of adapting to respond with the target chemical, wherein the viable bacterial cells can be monitored for stress imposed by the target chemical on the viable bacterial cells in absence of nutrient/culture medium.

In yet still another preferred embodiment, the present invention is directed to a method of detecting the presence, measuring the amount or verifying a target chemical of interest in a test sample, wherein the method is characterized using the bio-capacitor sensing device.

In yet still another embodiment, the present invention is directed to method of quantitatively detecting a target chemical of interest, the method comprising the steps of: exposing a test sample to the bio-capacitor device, wherein the test sample contains the target chemical of interest, whereby the test sample is capable of inducing a cellular stress response to the bio-capactitor device; applying a potential profile with an alternative current (AC) frequency to the bio-capacitor device; monitoring the cellular stress response of the bio-capacitor device by measuring the change in surface impedance/capacitance of the bio-capacitor device by nFEIS under no interfering nutrient/culture medium, wherein the cellular response correlates with the presence of the target chemical of interest.

It is well known to those skilled in the art that an impedance biochip can be divided into two groups: non-faradaic and faradaic. A non-faradaic biochip, here typically is known to as a capacitance biochip or a bio-capacitance chip and can be used interchangeably throughout the specification.

It is known to those skilled in the art that the effect of radiation at GHz frequencies on rat basophil leukemia cells is predominantly shown to be thermal in nature. Therefore, considering this effect, the stress responses with E. coli biochip sensor preferably was monitored only with the applied AC electrical frequencies below 600 MHz ensuring that no thermal effect occurred during the capacitance measurements. More preferably, the capacitive responses were observed at a frequency sweep from about 50 to about 600 MHz.

In still yet another preferred embodiment, the present invention is directed to a method of quantitatively detecting a target chemical of interest, the method comprising the steps of: exposing a test sample to the bio-capacitor device with no interfering nutrient/culture medium, wherein the test sample contains the target chemical of interest, whereby the test sample is capable of inducing a cellular stress response to the bio-capacitor device; applying a potential profile with an alternative current (AC) frequency to the bio-capacitor device; monitoring the cellular stress response of the bio-capacitor device by measuring the change in surface impedance/capacitance of the bio-capacitor device by nFEIS, wherein the cellular response correlates with the presence of the target chemical of interest under dry conditions with no interfering nutrient/culture medium.

In accordance to a preferred embodiment of the present invention, planar capacitor arrays are made of interdigitated microelectrodes preferably are pre-activated with carboxylated carbon nanotubes (CNTs) as opposed to, for example, electrode pairs placed in isolated culture vessels to contain liquid medium.

In accordance to a most preferred embodiment of the invention, the measurement of the cellular activity of bacteria present on the capacitor electrodes preferably is taken under dry conditions in absence of any nutrient/culture medium. Culture medium or any other liquid medium typically is known to alter the behavioral response of cells, thus making it difficult to distinguish the responses induced by the chemical agent in the context, from that of a complex mixture of other chemicals present in the nutrient medium. Previous methods in the art generally require that culture/liquid medium should be present in order to obtain a cellular response, however, this can interfere with actual response of a target chemical in the nutrient mixture.

In yet still another preferred embodiment of the present invention, the bacterial cells are covalently bonded on the CNTs present on the capacitors, as opposed to cells that are grown in dispersion or suspension or physically attached on electrodes.

According to the present invention, the capacitance or frequency change is measured as opposed to “resistance change” or “voltage change” to probe cellular activity.

Viable bacterial cells if coupled to electronic transducers, such as gold interdigitated (GID) capacitors (bio-capacitor) can deliver non-invasive cytotoxic information through cell-surface charge distribution and surface capacitance/impedance occurred by toxic chemicals at bacteria-capacitor interface provided, no interfering liquid nutrient/culture medium is present. The toxicity response of bacterial cells is often determined in terms of various stress responses. The stress responses in bacteria are classified into different types based on the nature of the chemical compound to induce toxicity. For example, chemicals that induce oxidative stress such as drugs that produce reactive oxygen species (ROS) and others induce osmotic stress, genotoxic stress, and other cellular stress responses.

Bacteria can respond to various cellular stresses under the alternative current (AC) electric field and the changes in electrical responses of bacteria to external stress can be captured or monitored by non-Faradaic electrochemical impedance spectroscopy (nFEIS) in dry conditions (no liquid nutrient/culture medium). According to an embodiment of the present invention, detection of the impact of toxic chemical by tethering live bacterial cells on GID electrodes as biological sensing surface (biochip). When toxic chemicals are exposed to the bacterial cells on sensor surface in absence of interfering nutrient/culture medium and drying, the cells responded to these chemicals and result in change in surface charge distribution that is measured by nFEIS against the AC electrical frequency sweep. As a result, the total charges present on the sensor/bacterial surface polarizes and relaxes that is dependent on a specific frequency. The change in response of bacterial cells on sensor surface against the toxic/stress chemicals can be monitored. The sensitivity of capacitor sensor surface is also enhanced by specific surface chemistries, including modifying with highly reactive nanomaterials, such as carbon nanotubes (CNTs), since CNTs possess unique structural, electronic and mechanical properties for a wide range of applications in electrochemical sensing.

According to a preferred embodiment of the present invention, a novel method is disclosed to detect toxicity of chemicals on viable bacteria to predict impact of such chemicals on humans complying with the ethical values to prevent using human cells. The proposed invention, according to embodiments, simplifies all of the problems associated with the previously available techniques for detection of toxicity induced by chemicals, by simply immobilizing bacterial cells on electronic capacitor chips in conjunction with carboxy-CNTs for signal enhancement. The toxicity detection or monitoring after a rapid and short exposure of dangerous chemicals, such as cancer causing chemicals (carcinogens), man-made chemicals (xenobiotics) is simply measuring the changes in surface capacitance/impedance in a rapid process at the capacitor-bacterial interface without actually harming the cells. In a preferred embodiment of the present invention, the bio-capacitor device gives away toxicity information of a suspected chemical within minutes that require no liquid nutrient/culture medium and also complying with the ethical regulations. This makes the bio-capacitor device more superior than the classical toxicity detection technologies. In addition, this bio-capacitor device has advantageous of being label free, are suitable due to small size and inexpensive.

It is known to those skilled in the art, that bacteria can respond to various cellular stresses under the AC electric field and the changes in electrical responses of bacteria to external stress can be captured or monitored by nFEIS. This can be accomplished by tethering live bacterial cells on GID electrodes as biological sensing surface (biochip). When toxic chemicals are exposed to these bacterial cells on sensor surface, the cells respond to these chemicals that result in surface charge distribution, which can be measured by nFEIS against the AC electrical frequency sweep. As a result, the total charges present on the sensor surface polarize and relaxes that depends on a specific frequency. The change in response of bacterial cells on sensor surface against the toxic/stress chemicals can be monitored. The sensitivity of sensor surface can be enhanced by various surface chemistries, including modifying with highly reactive nanomaterials, such as carbon nanotubes (CNTs) as these possess unique structural, electronic and mechanical properties that make them a very attractive material for a wide range of applications in electrochemical sensing. Recently, CNTs have been used as an electrode material for supercapacitors and also attracted much of attention because of their microscopic and macroscopic porous structures, electrochemical behavior, size and surface area that are important for abundance of reaction sites, and provides large-charge storage capacity and capacitance. CNTs exhibit space charge polarization at the electrode-nanotube interface under the applied ac-electrical frequency and possessing superior power densities due to fast charge/discharge capabilities.

In a preferred embodiment of the present invention, a novel method to determine toxicity of chemicals, that is, label-free and noninvasive approach that utilizes carboxy-CNT activated gold interdigitated capacitors immobilized with E. coli bacteria activated on GID capacitors as biosensing elements, and does not require participation of any mediators by nFEIS. According to an embodiment of the present invention, the sensitivity of sensor surface typically is enhanced by covalent activation on capacitors with carboxy-functionalized multiwalled CNTs that are typically less toxic than single walled CNTs. The toxicity behavior of toxic chemicals such as cancer causing chemicals (carcinogens) and man-made chemicals (xenobiotics) can be rapidly detected at the bacteria-capacitor interface of bio-capacitor. The proposed detection methodology is based on nFEIS, which can be used to screen various chemicals, toxic gases, pharmaceuticals, drugs, defense agents, environmental and food samples for the determination of chemicals' potential to cause cytotoxicity.

According to a typical embodiment of the present invention, the methods and device would be able to screen various chemicals, toxic gases, pharmaceuticals, drugs, defense agents, environmental and food samples for the determination of chemicals' potential to cause cytotoxicity.

According to an embodiment of the present invention, preferably, acetic acid, H₂O₂ and NaCl were employed as model chemicals to test the biochip and their responses were monitored under AC electrical field by nFEIS. The electrical properties of E. coli cells under different stresses were studied based on the change in surface capacitance as a function of applied frequency (300-600 MHz) in a label-free and noninvasive manner. The capacitive response of E. coli biochip under normal conditions exhibited characteristic dispersion peaks at 463 and 582 MHz frequencies. Deformation of these signature peaks determined the toxicity of chemicals to E. coli on capacitive biochip in the absence of liquid nutrient/culture medium. The E. coli cells were sensitive to, and severely affected by 166-498 mM (1-3%) acetic acid with declined capacitance responses. E. coli biochip exposed to H₂O₂ exhibited adaptive responses at lower concentrations (<2%), while at higher level (882 mM, 3%), the capacitance response declined due to oxidative toxicity in cells. However, E. coli cells were not severely affected by high NaCl levels (513-684 mM, 3-4%) as the cells tend to resist the salt stress. Our results demonstrated that the biochip response at a particular frequency enabled determining the severity of the stress imposed by chemicals and it can be potentially applied for monitoring unknown chemicals as an indicator of cytotoxicity.

In still yet a preferred embodiment of the present invention, a method of producing a bio-capacitor sensing device is disclosed. The method comprising the steps of: providing a substrate; depositing a metal layer on the substrate to form a capacitor, wherein the metal layer comprises at least one electrode; patterning the metal layer on the capacitor; attaching a layer of carboxylated carbon nanotubes (carboxy-CNTs) to the capacitor to form a carboxy-CNT activated capacitor; immobilizing viable cells to the carboxy-CNT activated capacitor, whereby the viable cells are sensing elements that are capable of adapting to respond with a target chemical, wherein the viable cells can be monitored for stress imposed by the target chemical on the viable cells.

Typically, the substrate is selected from the group consisting of silicon, glass, melted silica, and plastics. Preferably, the substrate is silicon.

According to an embodiment of the present invention, the electrode typically is an electrical conductive material, for instance, a material selected from the group consisting of gold, silver, platinum, palladium, copper and indium tin oxide (ITO). Preferably, the electrode is gold. The capacitor is a gold interdigitated capacitor.

The layer of carbon nanotubes preferably are carboxylated multiwalled carbon nanotubes (carboxy-CNTs).

According to an embodiment of the present invention, the viable cells typically can be selected from the group consisting of mammalian cells, bacterial cells and tissue cells of specific function. Preferably, the viable cells are bacterial cells comprising Escherichia coli, Salmonella and K-12. More preferably, the bacterial cells are Escherichia coli.

Patterning GID array electrodes patterning and fabrication of capacitor arrays. According to the present invention, the substrate can be selected from the group consisting of silicon, glass, melted silica and plastics. GID array electrodes were patterned on SiO₂ substrate surface using negative photolithography technique. In this process, the metal layers should be patterned using the dual tone photoresist AZ5214E. A 2 μm thick AZ5214E photo resist was patterned with the help of a mask for a lift-off process in pure acetone as a solvent. Following this step, a very thin tungsten layer of 50-60 nm size is layered to improve the adhesion of gold on the SiO₂ film by DC sputter deposition and about 200-210 nm thick gold layer was deposited. The dimension of each electrode should be 800 μm in length, 40 μm in width with a distance between two electrodes of 40 μm. Each capacitor sensor contains 24-interdigitated gold electrodes within a total area of 3 mm². The surface characterization is performed using Atomic Force Microscopy (AFM, Nanoscope) with the tapping mode and by optical micrographs.

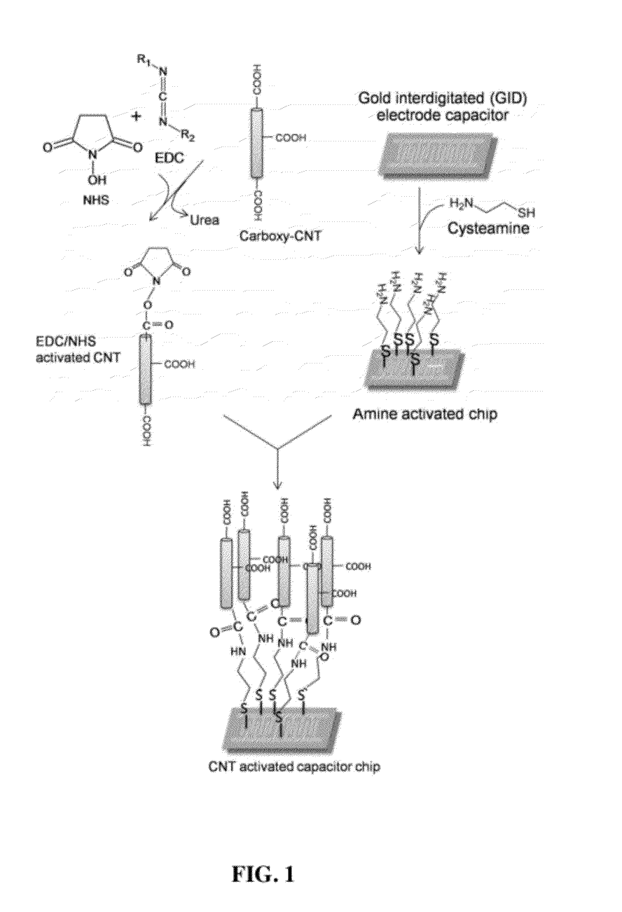

In an embodiment of the present invention, FIG. 1 illustrates an exemplary schematic diagram of activation of gold interdigitated electrode capacitor chip with carboxy-CNT fictionalization. According to FIG. 1, a method for immobilization of carboxy-CNTs on GID electrode capacitor arrays is shown according to an embodiment of the present invention.

Immobilization of carboxy-CNTs on GID electrode capacitor arrays. The interdigitated gold electrode array capacitive chip was subjected to plasma cleaning and thoroughly washed with ethanol and dried under a stream of N₂ gas.

The CNT which can be used in the present invention, is not particularly limited and can be commercially available products or prepared by any conventional method known to those skilled in the art. Typically, CNT should be carboxylated at its surface and/or both ends to be used in the present invention.

Any procedure known to those skilled in the art on covalent immobilization of carboxy-CNTs on capacitor chips can be used and incorporated herein by reference.

The bare GID electrode capacitor array chip immersed into a solution of 1 mM 95% cysteamine (Sigma-Aldrich) in ethanol for 24 h. The chip is removed and washed with ethanol and dried under a stream of N₂ gas. The self-assembled monolayer (SAM) of cysteamine formed on gold surface through —SH groups contained free —NH₂ groups that were utilized to covalently attach carboxylated multiwalled carbon nanotubes (carboxy-CNTs). For this, 100 μL of 1 mg/mL carboxy-CNTs (Arry®, Germany) in 99.9% dimethyl sulfoxide (Sigma-Aldrich) is mixed with equal volume of a mixture of 200 mM of 1-Ethyl-3-[3-dimethylaminopropyl]carbodiimide hydrochloride (EDC) and 100 mM N-hydroxysuccinimide (NHS) and ultrasonicated with alternative cycles of 10 s pulse after every 10 s interval for 5 min using ultrasonicator probe (Vibra cell 75043). The carboxy-CNTs suspension is incubated for 4 h at room temperature. About 5 μL of this suspension is dropped on each GID electrode covering an area of 3 mm² of each capacitor in an array of capacitors on SiO₂ wafer that were previously activated with cysteamine self-assembled monolayer. The capacitor chips were then incubated in airtight moist chamber for 24 h for covalent attachment of carboxy-CNTs. The capacitor arrays were then washed first with 50% DMSO in water followed by washing with acetone to remove traces of unbound carboxy-CNTs and dried over N₂ gas. A capacitor array without carboxy-CNT immobilization is used as a control for the comparison.

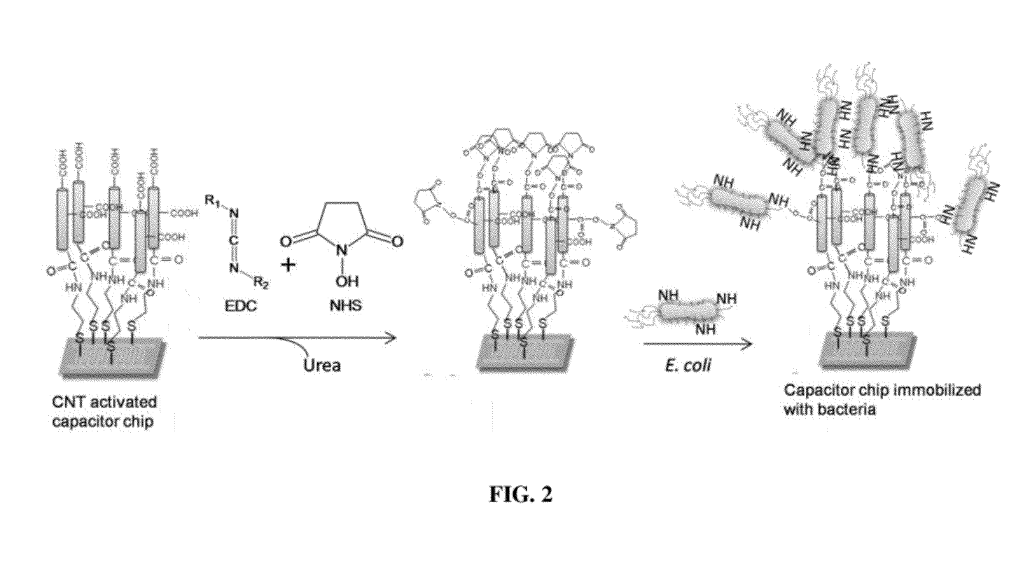

In an embodiment of the present invention, FIG. 2 illustrates an exemplary schematic diagram of biofunctionalization of carboxy-CNT activated GID capacitor chip and immobilization of E. coli cells to develop biochips. With respect to FIG. 2, a method for immobilization of E. coli cells on carboxy-CNTs activated GID capacitor arrays is shown according to a preferred embodiment of the present invention. Typically, any bacterial strain known to those skilled in the art can be used according to the present invention. Preferably, the bacterial strain is E. coli DH5α.

Method for immobilization of E. coli cells on carboxy-CNTs activated GID capacitor arrays. Actively growing E. coli cells were inoculated into fresh Luria Bertani (LB) medium and allowed to grow till mid-logarithmic growth phase. The cells were then harvested by centrifugation at 1000×g for 3 min and washed thrice with phosphate buffered saline (PBS) pH 7.2 and resuspended the cells in same buffer. The cell concentration is determined by colony counting after serial dilution followed by plating on LB-agar plates.

The carboxy-CNTs activated GID capacitor array chip is first rinsed in sterile distilled water and dried with pure nitrogen, and incubated with a mixture of 100 mM of EDC and 50 mM NHS for 2 h. The chip is then removed, thoroughly washed with distilled water and incubated with 5 μL of bacterial suspension containing two different concentrations of 8.7×10⁶ and 1.74×10⁷ colony forming units (CFU) in PBS buffer, respectively for 2 h. Optical micrographs were taken after immobilization of different concentration of bacterial cells.

Exposure of stress chemicals on GID capacitor chips immobilized with E. coli (biochip). The biochip was first washed with PBS buffer and preincubated at 37° C. for 1 h in moist chamber. To a series of capacitor arrays of biochip, 5 μL of different concentrations of three stress chemicals, such as acetic acid (0-498 mM), hydrogen peroxide (0-882 mM) and sodium chloride (0-684 mM) as models were incubated on each capacitor for 1 h and 3 h, respectively at 37° C. in an array of capacitors previously activated with carboxy-CNTs and immobilized with E. coli cells. Following this step, the biochip was washed with PBS buffer, pH 7.2, quickly dried to remove buffer and the moisture around the cells and the response of cells were directly monitored in absence of any liquid nutrient/culture medium.

Impedance/capacitance measurements. The impedance/capacitance responses were measured before and after the chemical treatment on the biochip surface by nFEIS. First, the capacitance/impedance were measured sequentially after every step that includes; (a) bare GID-capacitors (blank), (b) after activation with carboxy-CNTs (c) after bioconjugation with E. coli cells, and finally (d) after exposure of biochip against different stress chemicals with different concentrations and time.

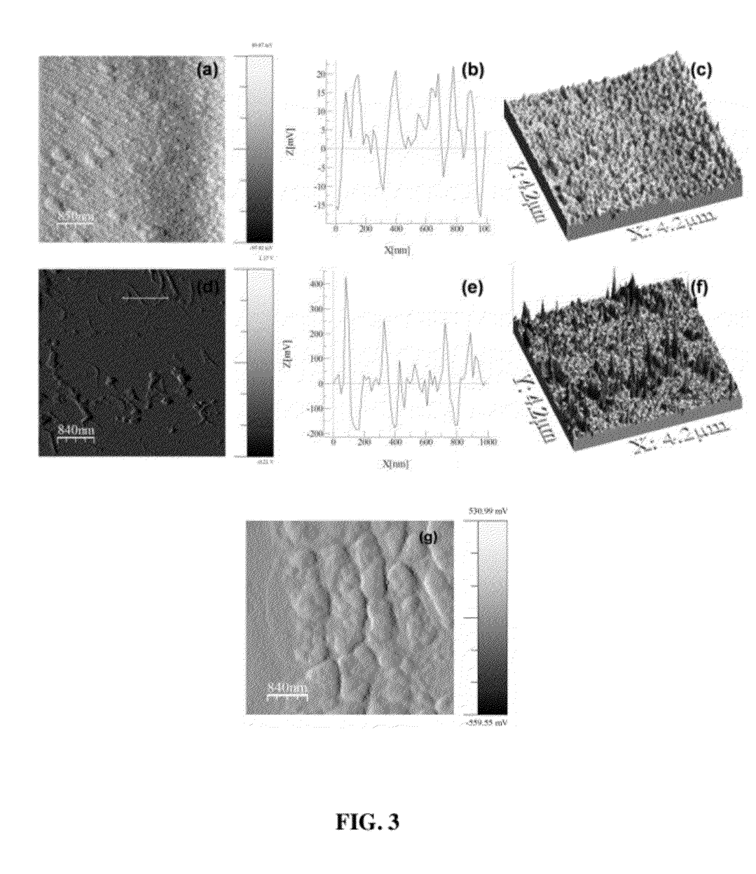

According to yet a preferred embodiment of the present invention, FIG. 3 illustrates tapping-mode AFM images (within 4.2×4.2 μm² scan area) of (a) bare GID surface, (b) Line plot surface profile of the selected green line region (1 μm length) in the tapping-mode AFM height image of bare GID surface, (c) 3D AFM topographical map of bare GID surface, (d) Tapping-mode AFM height image of GID surface activated with carboxy-CNTs, (e) Line plot surface profile of the selected green line region (1 μm length) in the tapping-mode AFM height image of GID electrode on capacitor surface activated with carboxy-CNTs, (f) 3D AFM topographical map of carboxy-CNT activated GID surface, and (g) A 2D tapping mode AFM image of a section (scan area 4.2 μm²) of biochip showing immobilized E. coli cells.

For control, biochips were treated with only PBS buffer in place of stress chemicals (blank) and a negative control experiment was conducted using biochip containing heat-killed E. coli cells. For this, biochips containing immobilized E. coli cells (1.74×10⁷) were subjected to heat treatment in an air-tight pre-heated humid chamber at 95° C. for 5 min followed by quickly freezing at −70° C. for 5 min, and thawed at 25° C. for 15 min. The above treatment process was repeated thrice and finally the biochip was dried under N₂ gas. The capacitance response in between the gold interdigitated electrodes was measured in the frequency range 50 MHz to 1 GHz using a Network Analyzer (Karl-Suss PM-5 RF Probe Station and Agilent-8720ES). The Network Analyzer was calibrated using SOLT (short-open-load-through) method. The impedance values were exported to MATLAB® software for the analysis. The absolute capacitance values of triplicate experiments were extracted at an effective frequency (f) range (300-600 MHz) and the standard deviations were shown as errors.

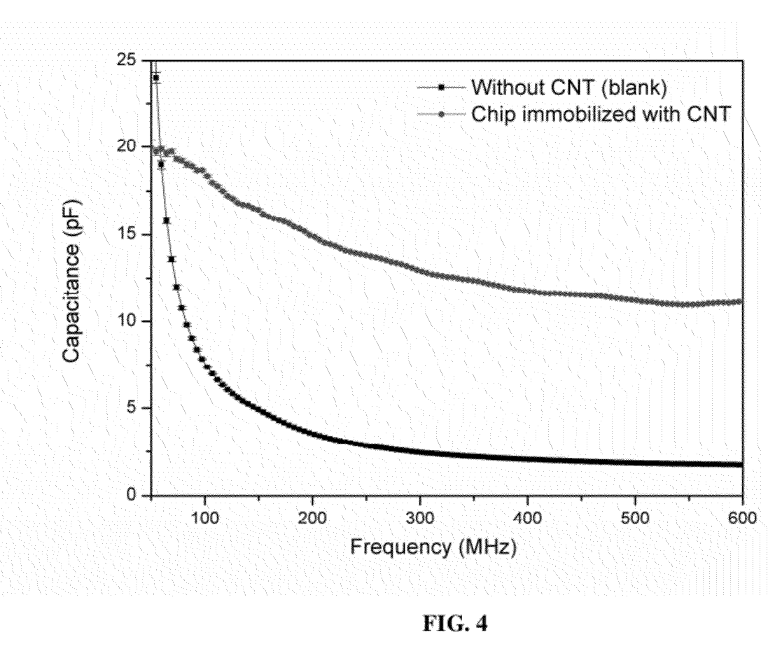

Characterization of biochip sensor surface by AFM image and optical micrographs. According to yet another preferred embodiment of the present invention, FIG. 4 illustrates an exemplary capacitive response of gold interdigitated capacitor chip before and after carboxy-CNT immobilization. Surface topographical AFM image of GID electrode capacitor before and after covalent attachment of carboxy-CNTs are shown in FIG. 3a-f. The bare GID electrode surface exhibited distribution of nanoparticles with varying diameter (˜100-200 nm) sizes (FIGS. 1a and b). AFM 3D height map image of the gold nanoparticles showed varying heights within scanned 4.2×4.2 μm² GID electrode surface area (FIG. 1c). The activated bare GID electrode surface by covalent immobilization of carboxy-CNTs was confirmed AFM images (FIG. 3d-f). After covalent immobilization of CNTs on the GID surface, the diameter of the carboxy-CNTs was determined to be varying from 50-70 nm (FIG. 3e). AFM 3D height map image of carboxy-CNTs activated GID electrode surface showed the distribution and varying heights of immobilized carboxy-CNTs. The response of bare GID capacitor surface was weakly charged and the activation of the sensor surface with carboxy-CNTs transformed into considerably highly charged surface (FIG. 4). The activated sensor surface was then subjected to biofunctionalization for the development of biochip.

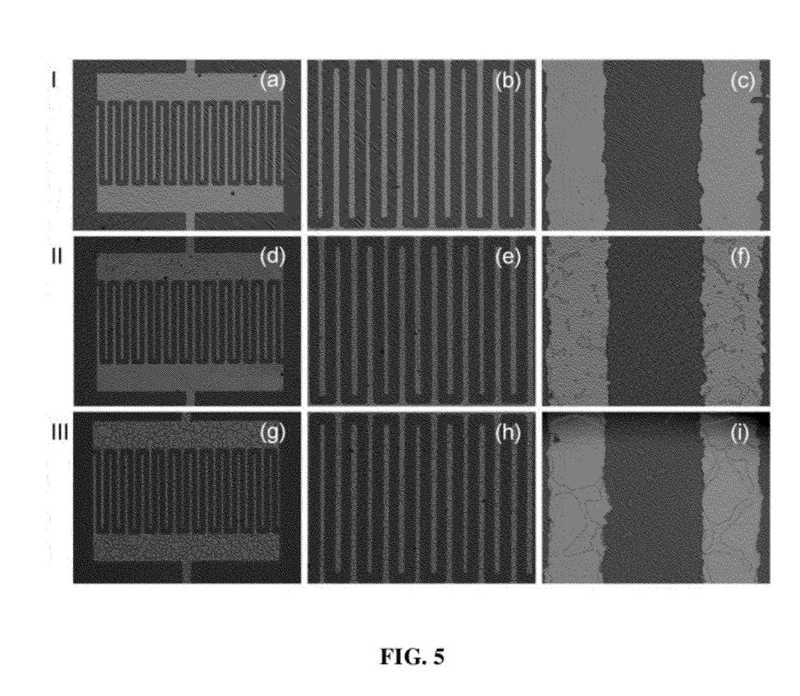

In yet another preferred embodiment of the present invention, FIG. 5 illustrates an exemplary optical micrographs of gold interdigitated capacitor surface: (I) activated with carboxy-CNTs (control), and carboxy-CNT activated chips immobilized with E. coli with concentrations of (II) 8.7×10⁶ cells and (III) 1.7×10⁷ cells. The rows (a-c, d-f and g-i) indicate optical resolutions of 5×, 10× and 100×, respectively.

The GID capacitor surface activated with carboxy-CNTs was biofunctionalized by immobilizing with two different concentrations of E. coli DH5α cells (8.7×10⁶ and 1.74×10⁷ CFU). Optical micrographs of carboxy-CNT activated electrode surface before and after immobilization of E. coli cells were examined (FIG. 5a-i). The microscopic observation of chips showed densely and covalently immobilized E. coli cells on GID surface. The non-specific adsorption on the SiO₂ surface of the chips was also observed, which consistently persisted even after repeated washing of chips with PBS buffer. Higher cell concentration resulted in densely immobilized gold electrode surface that was clearly distinguishable from the lower cell density immobilized (FIG. 5d-i). A 2D AFM image of a section of biochip showing immobilized E. coli cells is given in FIG. 3g.

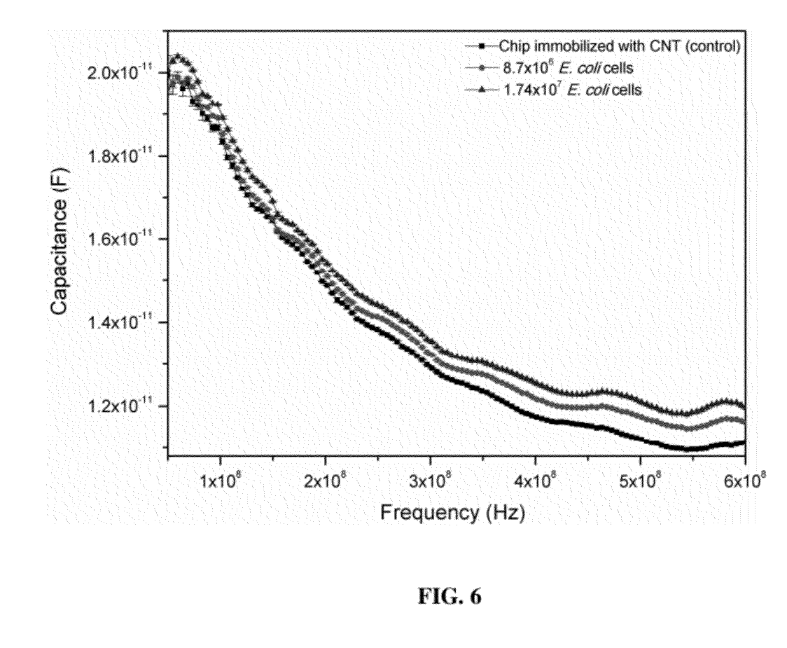

In still yet another embodiment of the present invention, FIG. 6 illustrates exemplary capacitive response of biochips immobilized with two different concentrations of E. coli cells on GID surface that was previously activated with carboxy-CNTs. The capacitive responses were observed at a frequency sweep of 50-600 MHz. The capacitance responses of viable E. coli cells were measured as a function of scanned AC electrical frequency (50 MHz to 1 GHz), and the cell density dependent increase in capacitance responses was observed (FIG. 6). At low frequency (50-200 MHz), the capacitance response was less dependent on cell concentration while it becomes more dependent on the cell concentration beyond 200 MHz of applied frequency (FIG. 6). Further, higher cell concentrations above 1.74×10⁷ CFU showed more non-specific adsorption on to SiO₂ surface. The cell concentration of 1.74×10⁷ CFU yielded considerably enhanced responses with minimum non-specific adsorption on SiO₂ surface, and this concentration was chosen to study the cell responses to different stresses under the AC electrical field. The capacitance decreased with the increasing frequency from 50 MHz to 600 MHz. In previous studies, the effect of radiation at GHz frequencies on rat basophil leukemia cells is predominantly shown to be thermal in nature. Therefore, the stress responses with E. coli biochip sensor was monitored only with the applied AC electrical frequencies below 600 MHz ensuring that no thermal effect occurred during the capacitance measurements. More preferably, the capacitive responses were observed at a frequency sweep from about 50 to about 600 MHz.

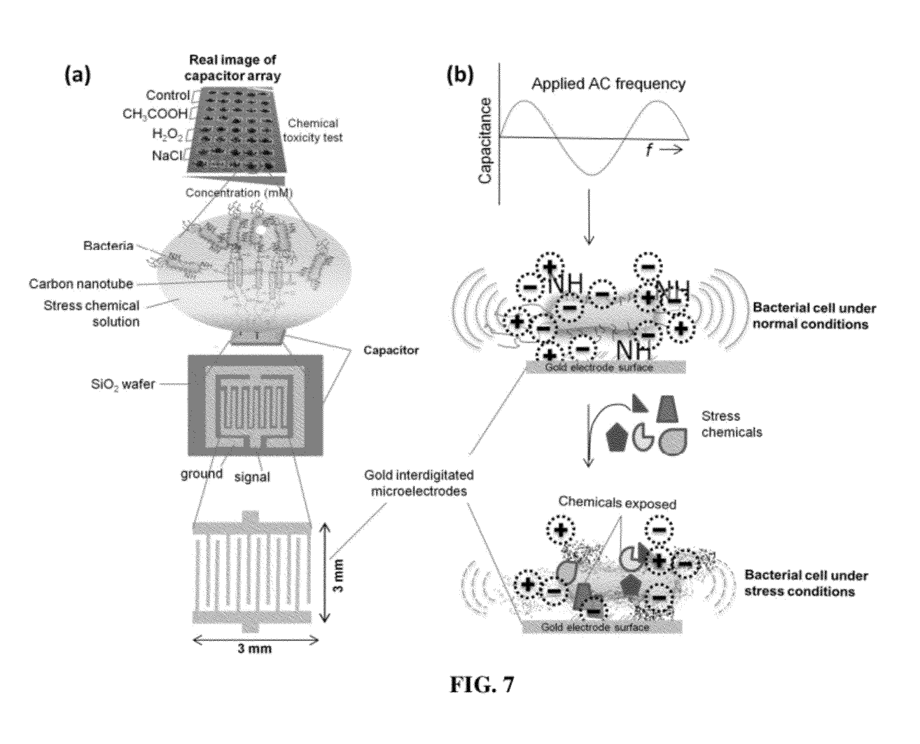

In yet another preferred embodiment of the present invention, FIG. 7 illustrates a schematic representation of (a) Capacitor array biochip immobilized with viable E. coli cells by tethering with carboxy-CNTs on gold interdigitated electrodes of each capacitor with a defined geometry and dimension; and (b) diagram showing the response of E. coli and surface charge distribution under the applied AC frequency in normal and chemical stress conditions.

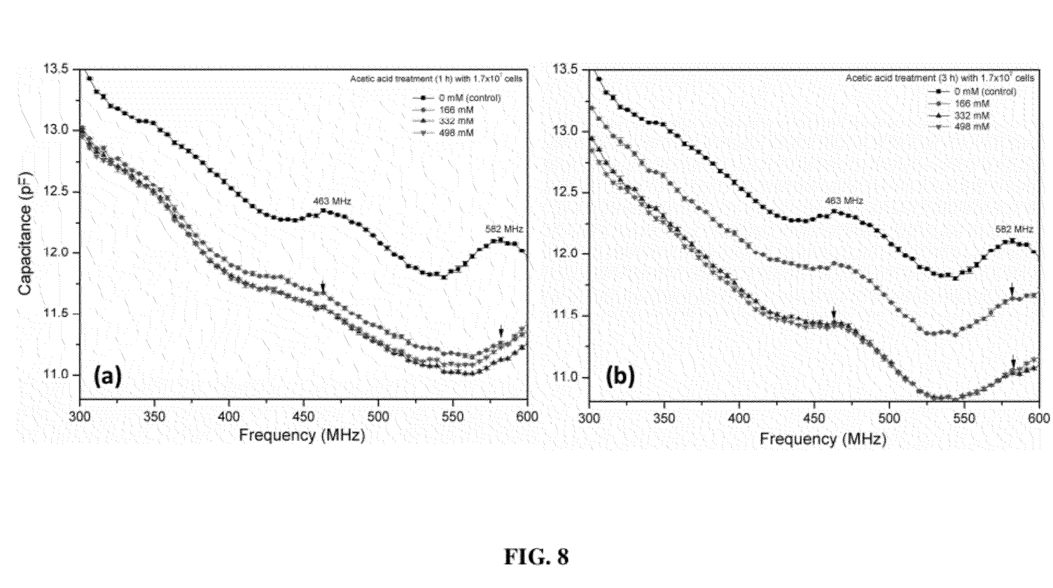

Electrical responses to chemical stresses by E. coli cells immobilized on biochip surface in absence of culture media (under dry condition). The carboxy-CNT activated capacitor surface immobilized with E. coli cells (1.74×10⁷ CFU) were treated with various concentrations of three model stress inducing chemicals, such as acetic acid (acid or metabolic stress), H₂O₂ (oxidative stress) and NaCl (salt stress) for 1 and 3 h. The chemical treated biochip was washed and gently dried before measuring the electrical responses to remove traces of moisture surrounding the cells on biochip. This allowed the chemicals that are bound or absorbed by cells to only be considered. The electrical responses of cells to different stresses were monitored using nFEIS after applying AC electrical frequency sweep from 300 to 600 MHz. The concentration and time dependent responses of cells to different stresses were analyzed in the context of only chemical-cell interactions, which is in absence of any liquid medium. Biochip responses were tested with concentrations of model chemicals above normal physiological levels that induce cellular stress responses. In yet still another preferred embodiment of the present invention, FIG. 8 illustrates a change in capacitance from E. coli capacitor biochip as a function of applied frequency (300-600 MHz) when exposed to different concentration of acetic acid for (a) 1 h and (b) 3 h according to the present invention. FIGS. 8a and b shows schematic diagram of arrays of capacitor biochip incubated with test chemicals and the response of E. coli cells with toxic chemicals.

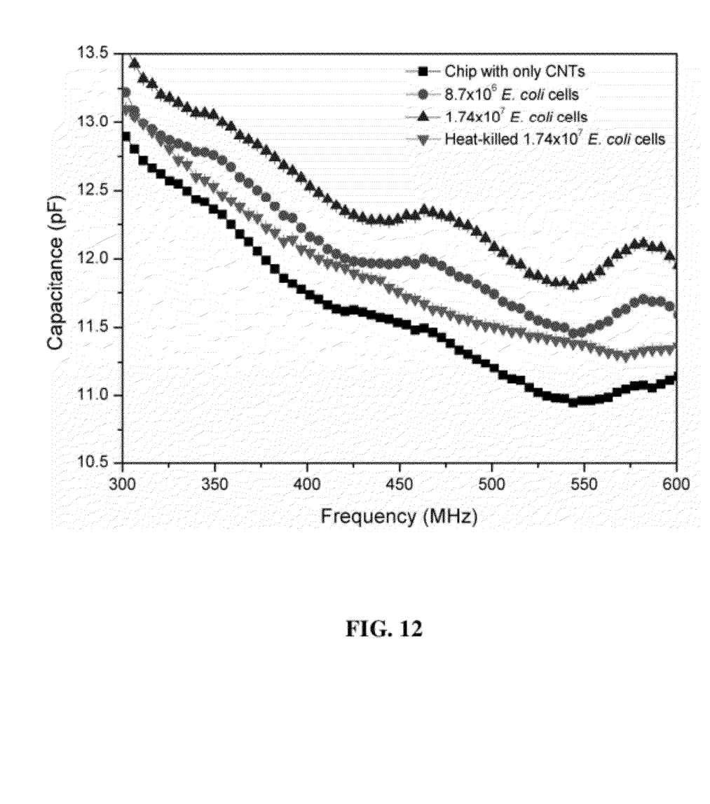

The biochip was first tested by applying AC electrical frequency sweep and extracted the data at an effective frequency (300-600 MHz). The capacitance response as a function of applied frequency yielded two specific dispersion peaks at 463 and 582 MHz frequencies under normal conditions (untreated cells) (FIG. 8a). An independent control experiment was conducted using biochip with heat-killed E. coli that did not show the characteristic dispersion peaks at 463 and 582 MHz, indicating that only viable cells exhibited the dispersion peaks (FIG. 12). These two peaks represented as a signature of cellular activity of immobilized E. coli under control/normal conditions.

The E. coli biochip was treated with different concentrations of acetic acid to probe the sensor responses. The characteristic dispersion peaks were diminished due to the stress imposed by acetic acid at initial 1 h treatment. The capacitive responses of the cells tended to decrease with increasing concentrations of acetic acid and its exposure time (1 h and 3 h) (FIGS. 8a and b). This decrease in capacitance can be attributed to the transport activity of acetic acid across the cell membrane combined with low pH of acetic acid that impaired the cell membrane function, which in turn may lower the cell's growth potential and viability.

The responses of cells were independent of acetic acid concentration at initial 1 h in lower frequencies (<350 MHz) while at higher frequencies the amplitude of capacitive response was distinguishable. The characteristic dispersion peaks at 463 and 582 MHz frequencies from the untreated cells (control) observed were diminished due to the acetic acid stress at initial 1 h treatment. However, after 3 h of acetic acid stress, the cells exhibited a distinct response pattern in which the concentration above 166 mM (1%) of acetic acid showed similar response with persistent dispersion peak at 463 MHz with diminishing 582 MHz peak (FIG. 8b). These results indicated that the cells tend to adapt to the stress exerted by the acetic acid over time (3 h) as evidence by the persistent dispersion peaks at 463 MHz and 582 MHz. After 3 h of acetic acid exposure, the cells were more sensitive to acetic acid above 166 mM (1%) unlike at initial 1 h exposure due to toxicity.

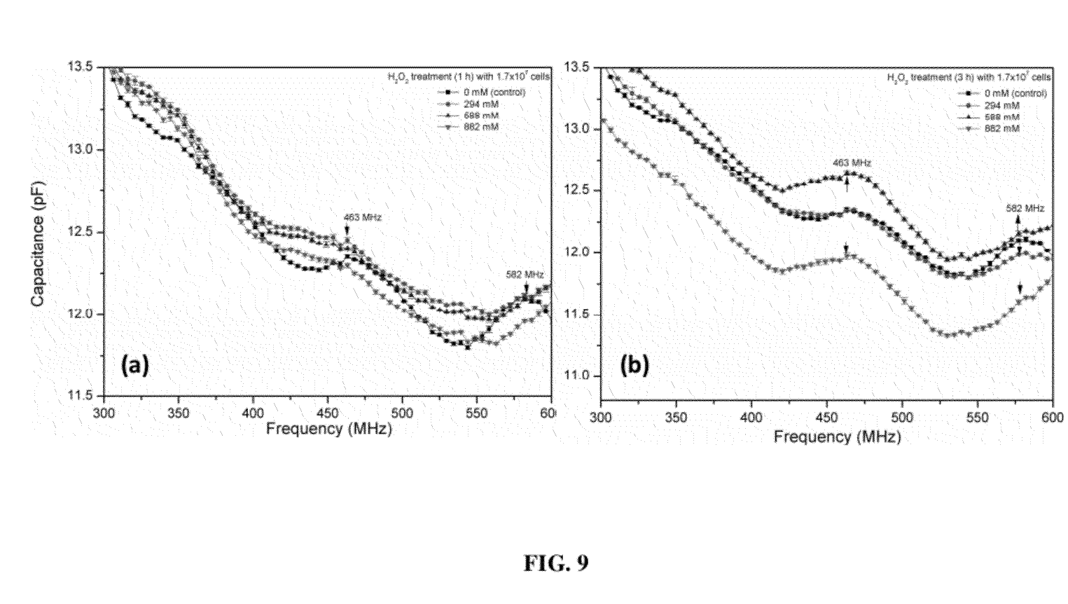

In yet another embodiment of the present invention, FIG. 9 illustrates a change in capacitance with E. coli capacitor biochip as a function of applied frequency (300-600 MHz) when exposed to different concentration of H₂O₂ for (a) 1 h and (b) 3 h. According to the present invention, E. coli biochip responses to oxidative stress were monitored. The biochip sensor surface was treated with different concentrations of H₂O₂ (0-882 mM, 0-3%) for 1 h and 3 h, respectively. E. coli cells treated with 294 mM (1%) of H₂O₂ resulted in a negligible change in capacitance response irrespective of exposure time but the disappearance of the characteristic dispersion peaks at 463 and 582 MHz was observed at 1 h, while they were persistent at 3 h, which is consistent to the fact that cells undergo adaptation to stress over 3 h time interval (FIGS. 9a and b). It was observed from FIG. 9b that the cells showed higher capacitance responses with 588 mM (2%) of H₂O₂ and this response tend to decline noticeably with 882 mM (3%) at 3 h of exposure with disappearing characteristic dispersion peak at 582 MHz. This result indicated that the E. coli cells resisted to low levels of oxidative toxicity that occurred by 294 mM (1%) H₂O₂. Whereas, at 588 mM (2%) H₂O₂ concentration, the cells displayed adaptive response to the oxidizing agent, which indicates that the exposure to low levels of H₂O₂ allows bacterial cells to resist exposure to further toxic doses of H₂O₂ (FIG. 9b). However, at even higher levels (882 mM, 3% of H₂O₂), the sensor chip exhibited considerably reduced capacitance responses, suggesting that the cells failed to cope up with the stress at higher level of oxidative toxicity caused by 882 mM (3%) as opposed to 588 mM (2%) of H₂O₂ at 3 h of exposure.

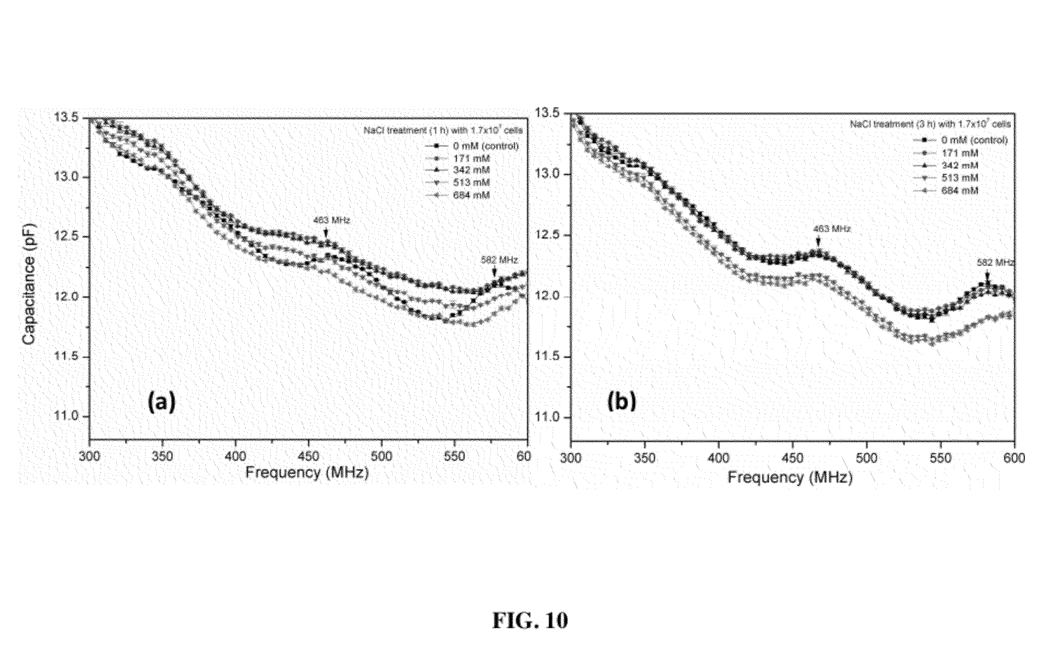

In yet still another preferred embodiment of the present invention, FIG. 10 illustrates change in capacitance from E. coli capacitor biochip as a function of applied frequency (300-600 MHz) when exposed to different concentration of NaCl for (a) 1 h and (b) 3 h. The CNT-activated sensor biochip was further tested by treating with yet another chemical, which is not toxic to cells, but at its higher levels can induce salt/osmotic stress. Various concentrations of NaCl (0-684 mM, 0-4%) was incubated on biochip to study the salt stress responses. The cells experienced mild adaptive responses to 0-513 mM NaCl (0-3%) concentration at the initial 1 h of treatment and the cells tend to experience the salt stress with 684 mM (4%) of NaCl concentration (FIG. 10a). In addition, at 1 h of salt exposure, the characteristic dispersion peaks were not prominent. At prolonged exposure of cells from 1 to 3 h, the salt concentration above 342 mM (2%) found to induce salt stress while the cells were not affected by the salt stress at lower concentrations (171-342 mM, 1-2%) (FIG. 10b).

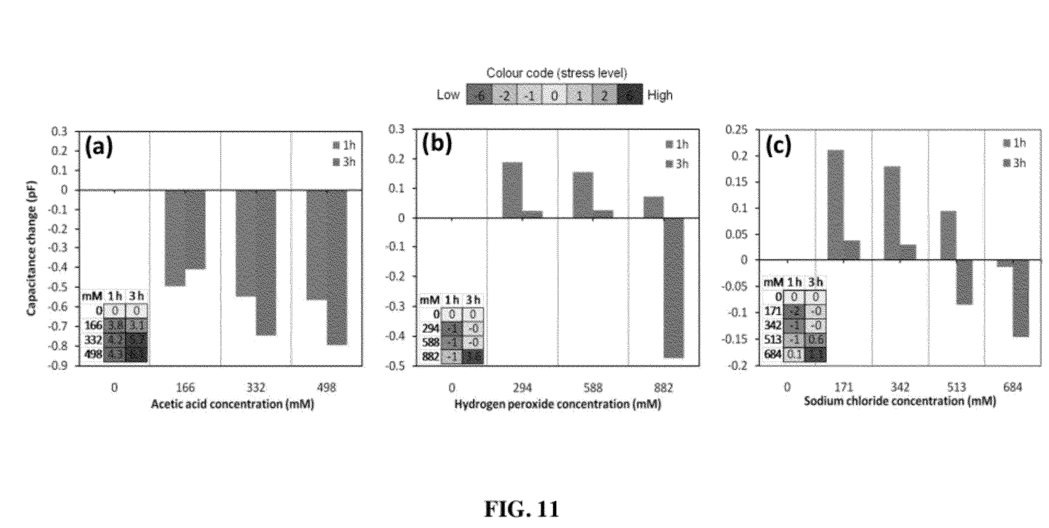

In yet still another embodiment of the present invention, FIG. 11 illustrates response of E. coli cells (immobilized on CNT activated sensor chip) as a function of different concentrations of (a) acetic acid (acid stress), (b) H₂O₂ (oxidative stress) and (c) NaCl (salt stress) at a constant AC electrical frequency (350 MHz) for 1 and 3 h treatment times. The inset tables show colour coded values determining the percent relative change in stress levels experienced by E. coli cells. The stress colour code scale indicates the severity of the stress levels in which green represent adaptation/resistance and red represent stress/toxicity.

Identifying the severity of stress imposed by chemicals and color coding. The severity of stress imposed by chemicals was color coded for visual inspection (FIG. 11). The colors red and green indicate toxic and non-toxic natures, respectively. The intensity of color indicates the severity of toxicity. For e.g., intense red indicates severe toxicity, while green indicates adaptive response. The results showed that treatment with acetic acid resulted in declining capacitance responses, which is an indicative of cells severely affected (shown red in the FIG. 11a) by the acetic acid over time (1-3 h). In contrast, H₂O₂ at initial 1 h exposure exhibited adaptive responses (shown green in FIG. 11b) while at 3 h at higher concentration (882 mM, 3%), the cells tend to be affected by oxidative stress imposed by high H₂O₂. FIG. 11b), suggesting that 882 mM (3%) H₂O₂ is toxic to cells. A similar response was observed with the cells treated with various salt concentrations. It was found that at initial 1 h of NaCl exposure, cells tend to adapt by increasing the surface charge distribution and thus increase in capacitance. However, after 3 h of exposure, the capacitance tends to decrease with higher levels above 342 mM (2%) of NaCl because of the salt stress (FIG. 11c). It is clear from the results that cells are not severely affected by NaCl compared to that seen with acetic acid or H₂O₂ indicating that cells can tolerate to high salt levels. This result is in agreement with previous studies where salt tolerance levels has been reported up to 1.1 M (˜6.5%) NaCl for E. coli and the cells tend to adapt to high salt concentrations for osmotolerance.

In another embodiment of the present invention, FIG. 12 illustrates exemplary results of capacitive response of bare GID surface covalently linked with only carboxy-CNTs (shown in black); biochip immobilized with viable 8.7×10⁶ cells (red) and 1.74×10⁷ cells (blue); and heat-killed 1.74×10⁷ cells (green) on GID surface that was previously activated with carboxy-CNTs. The capacitive responses were observed at a frequency range of 300-600 MHz.

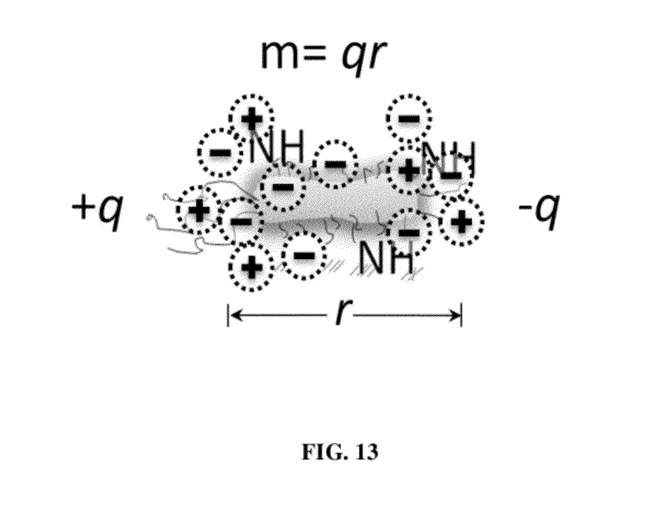

According to still yet another embodiment of the present invention, FIG. 13 illustrates an exemplary schematic diagram of a typical cell surrounded by a cloud of charges that constitutes a molecular dipole ‘m’ by two equal and opposite unit charges, separated by a distance ‘r’ on an outer cell surface. Results showed the altered behavior of bacterial cells to stress chemicals under AC electrical field by bacterial biochip. The underlying hypothesis of the developed E. coli based capacitive biosensor is as follows: a complex bacterial cell surface consists of positive and negative charges that are constituted from the ionizable side chains of surface and pili-proteins in the outer membrane. A typical bacterial cell, for example, a globular protein, typically exhibits surface charges that constitutes an electric dipole. The simplest molecular dipole of a bacterial cell (E. coli) typically consists of a pair of opposite electrical charges with magnitudes of +q and −q that are separated by a vector distance r. The molecular dipole moment m is given by the equation m=qr (FIG. 13). According to an embodiment of the present invention, a bacterial cell immobilized on a solid surface when exposed to any toxic chemical, the cells can experience a stressful condition since the outer membrane can becomes fragile or even disintegrated upon interaction with toxic chemicals, and thus exhibit altered surface charge distribution.

According to the present invention, a noninvasive, label-free capacitive biochip conjugated with viable E. coli cells (biochip) as biological recognition elements for determining the impact of chemicals to induce cellular stress by nFEIS was developed. According to the present invention, model chemicals were used to test the response of the E. coli biochip such as (a) acetic acid, which induces metabolic stress or acid shock, (b) hydrogen peroxide, contributes to oxidative toxicity through .OH generation, and (c) sodium chloride induces salt/osmotic stress. Distinct responses of the E. coli biochip to different chemicals in a concentration dependent manner provided knowledge on the toxicity of a given chemical.

Living cells can consist of a complex spatial arrangement of materials that have different electrical properties. Typically, bacteria have a cell membrane where oxidative phosphorylation occurs (in absence of mitochondria). It is known to those skilled in the art that the cell membrane of bacteria is surrounded by cell wall, which is rigid and protects the cell from osmotic lysis or external environmental purturbations. In Gram negative bacteria, the outer membrane is made of lipopolysaccharides and proteins. The cell membrane consists of a lipid bilayer containing many proteins, where the lipid molecules are oriented with their polar groups facing outwards into the aqueous environment, and their hydrophobic hydrocarbon chains pointing inwards to form the membrane interior. The inside of a cell contains membrane covered particulates and many dissolved charged molecules. While the cell membrane typically is highly insulating, the interior of the cell is highly conductive. The observed dielectric properties of bacterial cells can be explained on the basis of a model consisting of a conducting cytoplasmic core, contained by a thin insulating membrane, which in turn is surrounded by a porous conducting cell wall.

Typically, the sensitivity of capacitor sensor surface was tested using bare GID capacitor sensors. According to a preferred embodiment of the present invention, the sensitivity of these sensors was enhanced by activating the GID surface by carboxy-CNTs as described herein. It was observed that the level of capacitance response was enhanced by several orders of magnitude (FIG. 4). Therefore, all experiments were conducted using carboxy-CNTs activated sensor chips immobilized with E. coli. Here, immobilization of two E. coli concentrations (8.7×10⁶ and 1.74×10⁷) were chosen because at these concentrations there was minimum non-specific adsorption on to SiO₂ surface. Further increase in cell concentration only yielded more non-specific adsorption on the sensor surface. For this, an additional step may be required during the chip fabrication process to prevent from non-specific adsorption, for example, passivation of the SiO₂ surface by photoresist polymer such as SU-8, and leaving only the GID area open that may enable efficient immobilization of bacterial cells and enhance the sensitivity.

In yet still another embodiment of the present invention, although this method exemplifies using bacteria, this method can be extended to other cells (bacterial or mammalian) or tissues of specific function to probe the responses to external stimulus.

Typically, at low frequencies, the capacitance response was less dependent on cell concentration/stress while it becomes more dependent above 300 MHz of applied frequency. It is well known to those skilled in the art that cells exposed to AC electrical frequency field may result in the movement of layers of ions both inside and outside the surface of the cell wall effectively and becomes electrically polarized. This polarization typically takes the form of electrical charges that are created on external and interfacial surface (FIG. 7b), which influenced declining levels of capacitance beyond 300 MHz and thus relaxation behavior was observed (FIG. 8-10).

The characteristic dispersion peaks were observed from the untreated cells (control at 463 and 582 MHz frequencies) when exposed to AC electrical field (300-600 MHz). Appearance of these characteristic dispersion peaks is a clear indication of the presence/attachment of viable E. coli under control conditions. When cells treated with different stresses with acetic acid, H₂O₂ and NaCl, the cells surface interacted with these stresses and showed the sensitivity toward adaptive/detrimental responses. The interaction of bacterial cells with stress most likely yields a reduction of net surface charges and this resulted in disappearance of the characteristic dispersion peaks. This is most likely because, the loss of the cells' membrane function when exposed to stressed environment, and as a result, the membrane becomes more porous to ions, and also causes extra cytoplasmic protein misfolding. It was possible to monitor the response of biochip at a specific frequency as the response was dynamic to the frequency sweep from 300-600 MHz. Therefore, a frequency of 350 MHz was arbitrarily chosen and extracted the absolute capacitance values to elucidate the distinct responses of E. coli cells against model stress chemicals with respect to their concentration and time. The severity of stress imposed by chemicals was color coded for visual inspection (FIG. 11a-c).

The results showed that treatment with acetic acid resulted in declining capacitance responses, which is an indicative of cells severely affected (shown red in the FIG. 11a) by the acetic acid over time (1-3 h). In contrast, H₂O₂ at initial 1 h exposure exhibited adaptive responses (shown green in FIG. 6b) while at 3 h with 882 mM (3%), the cells affected by oxidative stress imposed by high H₂O₂ (FIG. 11b), suggesting that 882 mM (3%) H₂O₂ is toxic to cells. A similar response was observed with the cells treated with various salt concentrations. It was found that at initial 1 h of NaCl exposure, cells tend to adapt by increasing the surface charge distribution and thus increase in capacitance. However, after 3 h of exposure, the capacitance tends to decrease with higher levels above 342 mM (2%) of NaCl because of the salt stress (FIG. 11c). It is clear from these results that cells are not severely affected by NaCl compared to that seen with acetic acid or H₂O₂ suggesting that cells can tolerate to high salt levels but not to high acetic acid or H₂O₂. This result is in agreement with previous studies where salt tolerance levels has been reported up to 1.1 M (˜6.5%) NaCl for E. coli and the cells could adapt to high salt concentrations for osmotolerance.

Typically, E. coli are involved in the maintenance, adaptation and protection of the bacterial envelope in response to a variety of stressors. It is known to those skilled in the art that the envelope stress response in E. coli typically is regulated by the two-component system comprised of membrane localized proteins such as BaeS and BaeR (BaeSR regulon) that govern the adaptive responses through efflux of toxic compounds and protects from other envelope perturbants, through unidentified mechanisms.

In yet a preferred embodiment of the present invention, the behavior of E. coli cells as biological recognition elements on biochips for monitoring the impact of stress inducing chemicals in a noninvasive method.

The development of a novel method for the detection of toxicity of chemicals on biological systems, using carboxy-CNT activated GID capacitor array chips immobilized with E. coli. The detection methodology is based on nFEIS technique for monitoring the stress imposed by chemicals on bacterial cells. Using the biochip developed in this study is an effective means to understand and distinguish between toxicity of chemicals from non-toxic ones. The bio-capacitor sensing device provides a sensor platform that can also be used to characterize the target chemicals in nature including toxic gases to induce stress/toxic responses. Interesting feature of the developed E. coli biochip is the dispersion peaks at 463 and 582 MHz as signature of activity of E. coli and the change in capacitance levels at these frequencies would determine the toxic nature of stress chemical.

The previously described present invention has many advantages. One of the main advantages of using the developed biochip is that the non-specific signal can be avoided immediately after the treatment by simply washing away the biochip surface with appropriate buffer and measuring the capacitance under dry conditions or with no interfering liquid nutrient/culture medium. The detection methodology and biochip developed, therefore, finds advantages in testing different physical forms of stress agents (including gaseous, solid or liquid phase) making these methods and devices especially valuable and can be extended to determining potential toxicity and screening for monitoring environmental contaminants and food samples. However, there are few challenges that need to be addressed in order to improve sensitivity through (a) better design of gold interdigitated electrodes, (b) geometry, (c) an additional step of a deposit passivation layer formation on SiO₂ surface is required during chip fabrication to prevent from non-specific adsorption of cells on SiO₂ surface, (d) surface chemistry, (e) signal-to-noise ratio, and (f) bio-chemical assay conditions.

The description above and below and the drawings of the present document focus on one or more currently preferred embodiments of the present invention and also describe some exemplary optional features and/or alternative embodiments. The description and drawings are for the purpose of illustration and not limitation. Those of ordinary skill in the art would recognize variations, modifications, and alternatives. Such variations, modifications, and alternatives are also within the scope of the present invention. Section titles are terse and are for convenience only.

What is claimed is:

1. A bio-capacitor sensing device for the detection of a target chemical, the sensing device comprising:
a) a capacitor comprising a substrate and a metal deposit layer on the substrate;
b) a layer of carboxylated carbon nanotubes (carboxy-CNTs); and
c) viable cells, wherein the viable cells are immobilized to the layer of carbon nanotube (CNT),
whereby the viable cells are sensing elements that are capable of adapting to respond with the target chemical,
wherein the viable cells can be monitored for stress imposed by the target chemical on the viable cells.

2. The device of claim 1, wherein the substrate is selected from the group consisting of silicon, glass, melted silica, and plastics.

3. The device of claim 2 wherein the substrate is silicon.

4. The device of claim 1, wherein the metal deposit layer on the substrate comprises at least one electrode in interdigitated fingers structure.

5. The device claim 4, wherein in the electrode is a material selected from the group consisting of gold, silver, platinum, palladium, copper and indium tin oxide (ITO).

6. The device of claim 5, wherein the electrode is gold.

7. The device of claim 1, wherein the capacitor is a gold interdigitated capacitor.

8. The device of claim 1, wherein the layer of carbon nanotubes are carboxylated multiwalled carbon nanotubes (carboxy-CNTs).

9. The device of claim 1, wherein the viable cells can be selected from the group consisting of mammalian cells, bacterial cells and tissue cells of specific function.

10. The device of claim 9, wherein the viable cells are bacterial cells.

11. The device of claim 10, wherein the bacterial cells may be any strain of bacterial cells comprising Escherichia coli DH5α, K-12, Salmonella, Pseudomonas and Bacillus species.

12. The device of claim 11, wherein the bacterial cells are Escherichia coli.

13. The device of claim 1, wherein the target chemical can be selected from the group consisting of, acetic acid, lactic acid organic calcium salts, propionate, formate, drugs that influence intracellular accumulation of anions; oxidative toxicity induced by chemicals that produce reactive oxygen species (ROS), H₂O₂, hydroxyl radical (.OH), superoxide anion (O₂⁻), organic hydrogen peroxide (ROOH), peroxynitrite (OONO), nitric oxide (NO); osmotic stress induced by high concentrations of solutes, NaCl, osmolytes in the cytosol of cells; carnitine, trihalose, glycerol, sucrose, proline, mannitol, glycine-betain and others that induce genotoxic stress.

14. A method of quantitatively detecting a target chemical of interest, the method comprising the steps of:
a) exposing a test sample to the bio-capacitor device of claim 1, wherein the test sample contains the target chemical of interest only, whereby the test sample is capable of inducing a cellular stress response to the bio-capactitor device;
b) applying a potential profile with an alternative current (AC) frequency to the bio-capacitor device;
c) monitoring the cellular stress response of the bio-capacitor device by measuring the change in surface impedance/capacitance of the bio-capacitor device by nFEIS in absence of interfering liquid nutrient/culture medium, wherein the cellular response correlates with the presence of only the target chemical of interest.

15. The method of claim 14, wherein the target chemical is a stress agent selected from the group consisting of acetic acid, lactic acid organic calcium salts, propionate, formate, drugs that influence intracellular accumulation of anions; oxidative toxicity induced by chemicals that produce reactive oxygen species (ROS), H₂O₂, hydroxyl radical (.OH), superoxide anion (O₂⁻), organic hydrogen peroxide (ROOH), peroxynitrite (OONO), nitric oxide (NO); osmotic stress induced by high concentrations of solutes, NaCl, osmolytes in the cytosol of cells; carnitine, trihalose, glycerol, sucrose, proline, mannitol, glycine-betain and others that genotoxic stress.

16. A method of producing a bio-capacitor sensing device, the method comprising the steps of:
a) providing a substrate;
b) depositing a metal layer on the substrate to form a capacitor, wherein the metal layer comprises at least one electrode in interdigitated structure;
c) patterning the metal layer on the capacitor;
d) covalently attaching a layer of carboxylated carbon nanotubes (carboxy-CNTs) to the capacitor to form a carboxy-CNT activated capacitor;
e) immobilizing viable cells to the carboxy-CNT activated capacitor,
whereby the viable cells are sensing elements that are capable of adapting to respond with a target chemical,
wherein the viable cells can be monitored for stress imposed by the target chemical on the viable cells.

17. The method of claim 16, wherein the substrate is selected from the group consisting of silicon, glass, melted silica, and plastics.

18. The method of claim 16, wherein in the electrode is a material selected from the group consisting of gold, silver, platinum, palladium, copper and indium tin oxide (ITO).

19. The method of claim 16, wherein the capacitor is a gold interdigitated capacitor.

20. The method of claim 16, wherein the viable cells are Escherichia coli.
